(19) United States
(12) Patent Application Publication
(10) Pub. No.: US 2025/0376439 A1
(43) Pub. Date: Dec. 11, 2025

(54) SYSTEMS AND METHODS FOR MEMBRANE ENHANCED STEAM REFORMING WITH CARBON DIOXIDE UTILIZATION

(71) Applicant: Saudi Arabian Oil Company, Dhahran (SA)

(72) Inventors: Rakesh Kumar, Al Khobar (SA); Mohammad Abdur Rakib, Dhahran (SA); Aadesh Harale, Dhahran (SA); Zied Soua, Dhahran (SA)

(21) Appl. No.: 18/739,906

(22) Filed: Jun. 11, 2024

(51) Int. Cl. C07C 273/04; B01D 53/22; B01D 71/02; C01B 3/36; C01B 3/38; C01B 3/48; C01B 3/50; C01C 1/04; C07C 29/48; C07C 273/10; C07C 273/16

(52) U.S. Cl. CPC C07C 273/04; B01D 53/228; B01D 71/02231; C01B 3/36; C01B 3/382; C01B 3/48; C01B 3/505; C01C 1/0488; C07C 29/48; C07C 273/10; C07C 273/16; B01D 2256/16; B01D 2257/504; B01D 2257/80; C01B 2203/0233; C01B 2203/0244; C01B 2203/0283; C01B 2203/041; C01B 2203/0475; C01B 2203/0495; C01B 2203/061; C01B 2203/068; C01B 2203/1241; C01B 2203/141; C01B 2203/16

(57) ABSTRACT

A process includes feeding atmospheric air to an air separation unit to produce a flow of nitrogen and a flow of oxygen; combining the oxygen with a hydrocarbon flow and water in an auto-thermal reformer to produce a retentate stream to a membrane water gas shift reactor (M-WGSR); generating, from the retentate stream to the M-WGSR, a permeate stream from the M-WGSR that includes a first flow of carbon dioxide and a first combined flow of hydrogen and nitrogen; feeding a retentate stream to a membrane steam methane reformer (M-SMR) to produce a permeate stream from the M-SMR that includes a second flow of carbon dioxide and a second combined flow of hydrogen and nitrogen; feeding the first and second combined flows to an ammonia synthesis unit to produce ammonia; and feeding the first and second flows of carbon dioxide and the ammonia to a urea synthesis unit to produce a flow of urea by fully utilizing the carbon dioxide.

### TECHNICAL FIELD

The present disclosure describes systems and methods for membrane enhanced steam reforming with carbon dioxide utilization.

### BACKGROUND

Ammonia is used worldwide in the production of fertilizer and raw material for industrial chemicals. The conventional approach to ammonia synthesis for urea and fertilizer includes a reaction of hydrogen with nitrogen in stoichiometric amounts using a suitable catalyst. Hydrogen generation for ammonia synthesis typically involves a combination of one or more processes among autothermal reforming and steam reforming to obtain a gas mixture of carbon monoxide, carbon dioxide, hydrogen, and water followed by carbon monoxide conversion in a two-stage catalytic water gas shift reactor. Conventional ammonia synthesis processes produce a substantial amount of carbon dioxide, which requires additional steps of purifications for its further utilization.

### SUMMARY

In an example implementation, a process includes feeding a flow atmospheric air to an air separation unit to produce a flow of nitrogen and a flow of oxygen; combining the flow of oxygen with a hydrocarbon flow and a flow of water in an auto-thermal reformer to produce a retentate stream to a membrane water gas shift reactor (M-WGSR); generating, from the retentate stream to the M-WGSR, a permeate stream from the M-WGSR that includes a first flow of carbon dioxide and a first combined flow of hydrogen and nitrogen; feeding a retentate stream to a membrane steam methane reformer (M-SMR) to produce a permeate stream from the M-SMR that includes a second flow of carbon dioxide and a second combined flow of hydrogen and nitrogen; feeding the first and second combined flows of hydrogen and nitrogen to an ammonia synthesis unit to produce a flow of ammonia; and feeding the first and second flows of carbon dioxide and the flow of ammonia to a urea synthesis unit to produce a flow of urea by fully utilizing the first and second flows of carbon dioxide.

In an aspect combinable with the example implementation, the retentate stream to the M-SMR includes another flow of water and another hydrocarbon flow.

In another aspect combinable one, some, or all of the previous aspects, the M-SMR is a first M-SMR, and the process further includes feeding a retentate stream to a second M-SMR to produce a permeate stream from the second M-SMR that includes a flow of hydrogen and a third flow of carbon dioxide.

Another aspect combinable one, some, or all of the previous aspects further includes combining the flow of hydrogen with the flow of nitrogen from the air separation unit into a third combined flow of hydrogen and nitrogen; and feeding the third combined flow of hydrogen and nitrogen to the ammonia synthesis unit to produce the flow of ammonia.

Another aspect combinable one, some, or all of the previous aspects further includes feeding a portion of the third flow of carbon dioxide from the second M-SMR to the urea synthesis unit to produce the flow of urea by fully utilizing the first, second, and portion of the third flows of carbon dioxide.

Another aspect combinable one, some, or all of the previous aspects further includes feeding another portion of the third flow of carbon dioxide from the second M-SMR and the flow of hydrogen from the second M-SMR to a methanol synthesis unit to produce a flow of methanol.

Another aspect combinable one, some, or all of the previous aspects further includes feeding a flow of steam to the second M-SMR as a sweep gas to produce the permeate stream from the second M-SMR.

Another aspect combinable one, some, or all of the previous aspects further includes feeding a portion of the flow of nitrogen to the M-SMR as a sweep gas to produce the permeate stream from the M-SMR; and feeding another portion of the flow of nitrogen to the M-WGSR as a sweep gas to produce the permeate stream from the M-WGSR.

Another aspect combinable one, some, or all of the previous aspects further includes outputting a portion of the flow of ammonia.

Another aspect combinable one, some, or all of the previous aspects further includes, in each of the M-SMR and the M-WGSR, utilizing a hydrogen selective membrane to produce the respective permeate streams from the M-SMR and M-WGSR.

In another example implementation, a system includes an air separation unit; an auto-thermal reformer in fluid communication with the air-separation unit; a membrane water gas shift reactor (M-WGSR) fluidly coupled to the air separation unit and the auto-thermal reformer; a membrane steam methane reformer (M-SMR) fluidly coupled to the air separation unit; an ammonia synthesis unit fluidly coupled to the M-WGSR and M-SMR; a urea synthesis unit fluidly coupled to the M-WGSR, the M-SMR, and the ammonia synthesis unit; and a flow control system configured to perform operations. The operations includes feeding a flow atmospheric air to the air separation unit to produce a flow of nitrogen and a flow of oxygen; combining the flow of oxygen with a hydrocarbon flow and a flow of water in the auto-thermal reformer to produce a retentate stream to the M-WGSR; generating, from the retentate stream to the M-WGSR, a permeate stream from the M-WGSR that includes a first flow of carbon dioxide and a first combined flow of hydrogen and nitrogen; feeding a retentate stream to the M-SMR to produce a permeate stream from the M-SMR that includes a second flow of carbon dioxide and a second combined flow of hydrogen and nitrogen; feeding the first and second combined flows of hydrogen and nitrogen to the ammonia synthesis unit to produce a flow of ammonia; and feeding the first and second flows of carbon dioxide and the flow of ammonia to the urea synthesis unit to produce a flow of urea by fully utilizing the first and second flows of carbon dioxide.

In an aspect combinable with the example implementation, the retentate stream to the M-SMR includes another flow of water and another hydrocarbon flow.

In another aspect combinable one, some, or all of the previous aspects, the M-SMR is a first M-SMR, the system includes a second M-SMR, and the operations further include feeding a retentate stream to the second M-SMR to produce a permeate stream from the second M-SMR that includes a flow of hydrogen and a third flow of carbon dioxide.

In another aspect combinable one, some, or all of the previous aspects, the operations further include combining the flow of hydrogen with the flow of nitrogen from the air separation unit into a third combined flow of hydrogen and nitrogen; and feeding the third combined flow of hydrogen and nitrogen to the ammonia synthesis unit to produce the flow of ammonia.

In another aspect combinable one, some, or all of the previous aspects, the operations further include feeding a portion of the third flow of carbon dioxide from the second M-SMR to the urea synthesis unit to produce the flow of urea by fully utilizing the first, second, and portion of the third flows of carbon dioxide.

In another aspect combinable one, some, or all of the previous aspects, the operations further include feeding another portion of the third flow of carbon dioxide from the second M-SMR and the flow of hydrogen from the second M-SMR to a methanol synthesis unit to produce a flow of methanol.

In another aspect combinable one, some, or all of the previous aspects, the operations further include feeding a flow of steam to the second M-SMR as a sweep gas to produce the permeate stream from the second M-SMR.

In another aspect combinable one, some, or all of the previous aspects, the operations further include feeding a portion of the flow of nitrogen to the M-SMR as a sweep gas to produce the permeate stream from the M-SMR; and feeding another portion of the flow of nitrogen to the M-WGSR as a sweep gas to produce the permeate stream from the M-WGSR.

In another aspect combinable one, some, or all of the previous aspects, the operations further include outputting a portion of the flow of ammonia.

In another aspect combinable one, some, or all of the previous aspects, each of the M-SMR and the M-WGSR includes a hydrogen selective membrane configured to produce the respective permeate streams from the M-SMR and M-WGSR.

Implementations of systems and methods for membrane enhanced steam reforming with carbon dioxide utilization according to the present disclosure can include one, some, or all of the following features. For example, implementations of the present disclosure can provide for an efficient hydrocarbon and carbon monoxide conversion to hydrogen using one or more membrane reactors in combination with an auto-thermal reformer and membrane water gas shift reactor in parallel with a membrane reformer. As another example, implementations of the present disclosure can avoid additional infrastructure otherwise requiring additional steam generation, by using nitrogen as a sweep gas. Also, implementations of the present disclosure can produce a permeate product from one or more membrane reactors that contains a mixture of hydrogen and nitrogen in a desired mole ratio, which can be used directly for ammonia production in an ammonia synthesis unit. Further, implementations of the present disclosure can enable co-production of ammonia, urea, and methanol with the complete utilization of carbon dioxide.

The details of one or more implementations of the subject matter described in this disclosure are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a schematic drawing of an example implementation of a process for membrane enhanced steam reforming with carbon dioxide utilization according to the present disclosure.

FIG. 2 is a schematic drawing of an example implementation of a membrane reactor that can be used in a process for membrane enhanced steam reforming with carbon dioxide utilization according to the present disclosure.

FIG. 3 is a schematic drawing of another example implementation of a process for membrane enhanced steam reforming with carbon dioxide utilization according to the present disclosure.

FIGS. 4 and 5 show tables of example process mole flows and mole fractions of fluid streams in the respective processes of FIGS. 1 and 3, respectively, according to the present disclosure.

FIG. 6 shows a schematic drawing of a control system that can be used to perform control operations for processes for membrane enhanced steam reforming with carbon dioxide utilization according to the present disclosure.

### DETAILED DESCRIPTION

The present disclosure describes example implementations of systems, methods, and processes that utilize a membrane enhanced hydrogen production process for ammonia synthesis with full (in other words, 100% or substantially 100%) utilization of a co-product of carbon dioxide (CO₂) in producing urea and methanol. By achieving full utilization of the CO₂, this greenhouse gas is not emitted into the atmosphere.

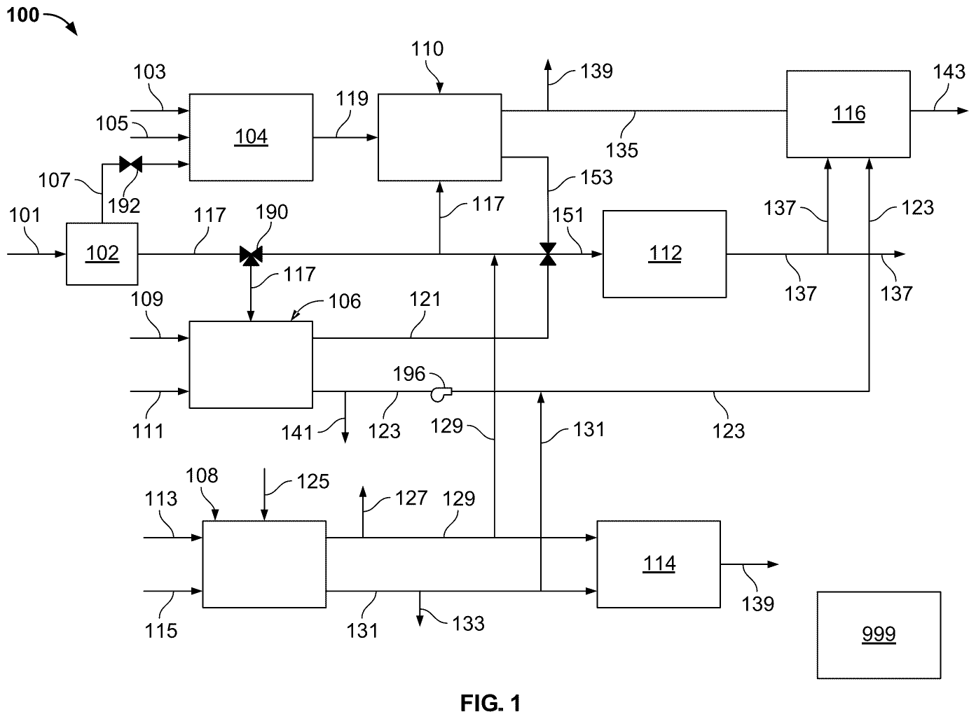

FIG. 1 is a schematic drawing of an example implementation of a process 100 for membrane enhanced steam reforming with carbon dioxide utilization according to the present disclosure. Generally, process 100 describes a system and process in which produces hydrogen and CO₂ by steam reforming of a hydrocarbon in an auto-thermal reformer (ATR) and dual membrane steam methane reformers (M-SMR) in a parallel configuration. Residual carbon monoxide (CO) is converted in a membrane water gas shift reactor (M-WGSR). Oxygen used for the ATR is produced by splitting air (for example, atmospheric air) to oxygen and nitrogen in an air separation unit (ASU). Nitrogen produced in the ASU can be used as a sweep gas in one of the dual M-SMRs, as well as the M-WGSR, while steam can be used as a sweep gas in another of the dual M-SMRs. Generally, the reactors comprise the respective steam reforming or water gas shift reaction catalysts and each is integrated with a hydrogen selective membrane.

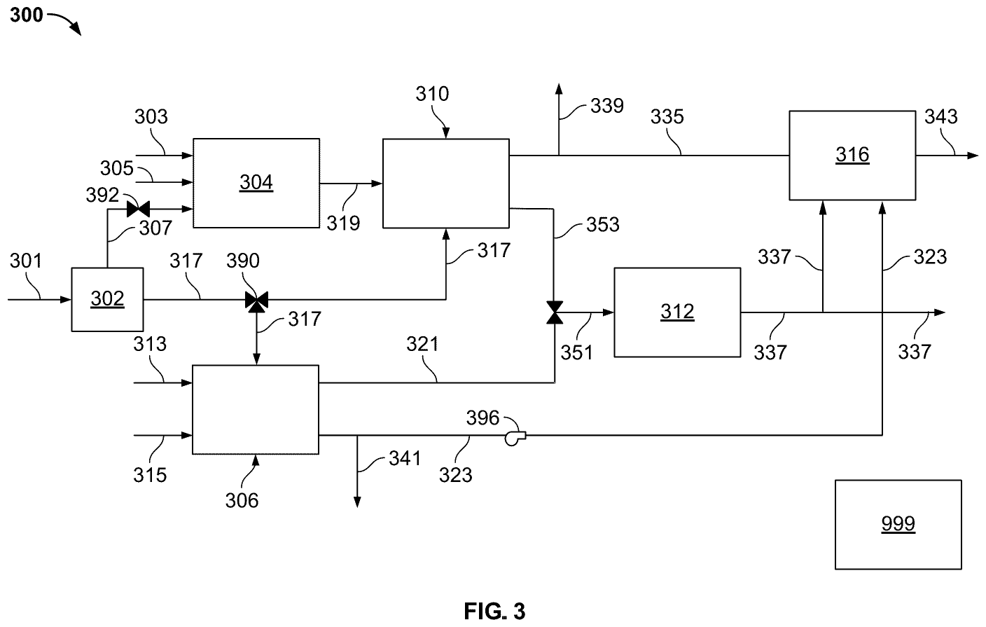

In process 100, a second membrane reformer is operated with steam as a sweep gas for the production of methanol and to balance the H₂ and CO₂ in ammonia and urea production. In case of ammonia and urea only production (in the example of FIG. 3), hydrocarbon is processed in ATR with a single membrane reformer in parallel with the M-WGSR. In both example processes 100 and 300 (described herein), ammonia is then produced in an ammonia synthesis unit. Ammonia and the separated CO₂ and are further converted to urea in in a urea production unit.

The process 100 includes process fluid flows that are circulated (naturally or forcibly or both) through the components of the process from left to right as shown in FIG. 1. Air 101 (for example, atmospheric air) is fed to an ASU 102 that separates the air 101 into oxygen 107 (O₂) and nitrogen 117 (N₂). The oxygen 107 is fed to an ATR 104 along with water 103 (H₂O) and a hydrocarbon 105 (such as, for example, natural gas 105). The nitrogen 117 is also provided to a first M-SMR 106, along with water 109 (H₂O) and a hydrocarbon 111 (such as natural gas). In this example, the nitrogen 117 is used as the sweep gas in the M-SMR 106. Water 113 (H₂O) and a hydrocarbon 115 (such as, for example, natural gas 115), along with steam 125, is provided to a second M-SMR 108. In this example, the steam 125 is used as the sweep gas in the M-SMR 108.

The ATR 104, in this example, can be a combination of steam methane reforming (SMR) and partial oxidation (POₓ), where the reactor comprises a combustion section to generate the heat required for the endothermic reforming reaction, which is carried out in a catalyst bed in the ATR 104. The ATR product 119 (for example, a product of hydrogen, carbon monoxide, carbon dioxide, methane, and water) is further processed in the M-WGSR 110. The ATR product 119, in some aspects, can have mole fractions of: 0.51 hydrogen, 0.19 carbon monoxide, 0.05 carbon dioxide, 0.01 methane, and 0.25 water. Nitrogen 117 from the ASU 102 is also provided to the M-WGSR 110.

The M-SMR 106 produces the combined flow 121 of H₂ and N₂, as well as carbon dioxide 123 (CO₂) and impurities 141. The M-SMR 108 produces hydrogen 129 (H₂) and carbon dioxide 131 (CO₂), as well as steam 127 and more impurities 133. In some aspects, carbon dioxide 123 is a carbon dioxide gas stream (CO₂) that emanates at a high pressure, which can be compressed (for example, by one or more compressors rather than pump 196) to circulate the carbon dioxide 123.

As shown in FIG. 1, the hydrogen 129 is combined with the nitrogen 117 (and joined with combined flow 121) to form the combined flows 121 and 151 of hydrogen (H₂) and nitrogen (N₂). The flow 151 is fed to an ammonia synthesis unit 112, which produces ammonia 137 (NH₃). Ammonia 137 is fed to a urea synthesis unit 116 as well as output from the process 100 as shown.

The hydrogen 129 (H₂) and carbon dioxide 131 (CO₂) from the M-SMR 108 are both fed to a methanol synthesis unit 114, which produces methanol 139 as an output from the process 100. The carbon dioxide 131 is also provided to combine with the carbon dioxide 123 that flows from the M-SMR 106; the combined flows of 123 and 131 are provided as carbon dioxide 123 to the urea synthesis unit 116.

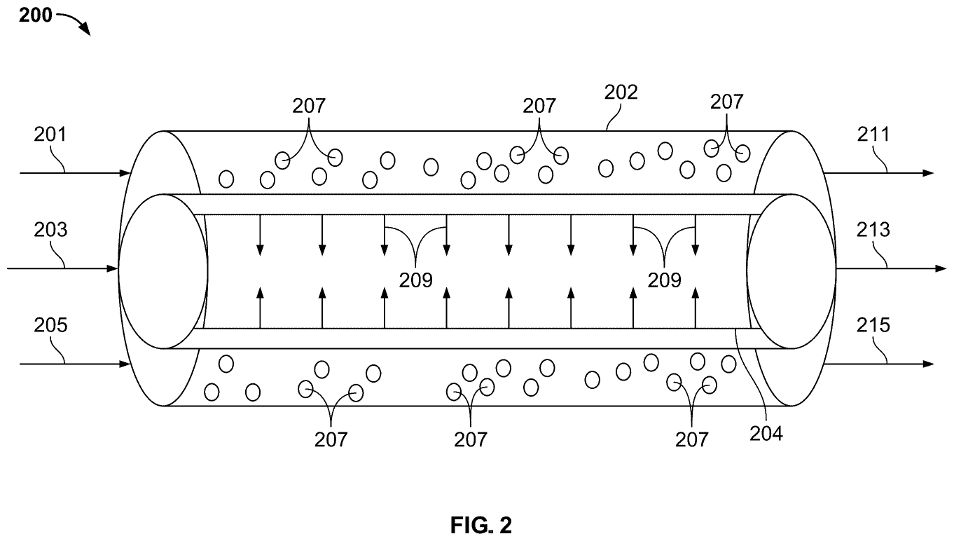

The M-WGSR 110 produces carbon dioxide 135 (CO₂) as well as a combined flow 153 of hydrogen (H₂) and nitrogen (N₂). The M-WGSR 110 also produces impurities 139. The combined flow 153 combines with the combined flow 121 and feeds into the ammonia synthesis unit 112 as flow 151. The M-WGSR 110 feeds the carbon dioxide 135 to the urea synthesis unit 116. In some aspects, the M-WGSR 110 contains a tubular hydrogen separation palladium alloy membrane (shown in more detail in FIG. 2). The carbon dioxide 135 and water are obtained as retentate product in the M-WGSR 110, which are further used as feed for urea and methanol production as shown.

With the flows 123 and 135 of carbon dioxide, as well as the ammonia 137, the urea synthesis unit 116 produces urea 143 as an output of the process 100. As shown in FIG. 1, therefore, in this example implementation, the only primary outputs of process 100 are urea 143, ammonia 137, and methanol 139, with all carbon dioxide 135 and 123 being consumed in the process by stoichiometric balancing. For example, the stoichiometric balancing can include providing the ammonia 137 and carbon dioxide 135 and 123 (combined) to the urea synthesis unit 116 in a molar ratio in the range of between 1 and 6 (urea to carbon dioxide).

Secondary outputs such as steam 127 and impurities 139, 141, and 133 are also produced. In this example, impurities can include carbon monoxide (CO) and methane (CH₄), which can be recycled back to the M-SMRS 106 and 108 and the M-WGSR 110. In some aspects, the impurity composition on a molar basis for the recycled impurities can be 2% for methane and 1.7% for carbon monoxide.

In the example process 100, the nitrogen 117 is used as a sweep gas to the M-SMR 106 in a co-current/counter current mode of operation for further enhancement of hydrocarbon and carbon monoxide conversion. Hydrogen and nitrogen in combined flows 153 and 121 are obtained as a permeate stream (for example, in the ratio of 3:1) from the M-WGSR 110 and M-SMR 106, respectively, which are further processed in the ammonia synthesis unit 112 for ammonia production. The mole ratio of hydrogen and nitrogen in the combined flows 121 and 153 can be adjusted by changing the sweep gas flow or by changing other operating and design parameters (for example, membrane area, permeate pressure, feed pressure and temperature) to obtain a desired mole ratio for ammonia synthesis (for example, between 2.7 and 3.2).

The process streams of the present disclosure can be flowed using one or more flow control systems 999 (e.g., FIGS. 1 and 3) implemented throughout the illustrated membrane enhanced hydrogen production processes shown in the present disclosure. A flow control system 999 can include one or more flow pumps (an example of which is shown in FIG. 1 as pump 196 and in FIG. 3 as pump 396) to pump the process streams, one or more flow pipes through which the process streams are flowed and one or more valves to regulate the flow of streams through the pipes. In the present disclosure, a “pump” or “flow pump” can refer to a liquid pump that forcibly circulates a liquid or mixed phase fluid, a fan that circulates a gas, a compressor that compresses and circulates a fluid, or a turbine that expands and circulates a fluid.

Control system 999 can include one or more monitoring devices. In example implementations, control system 999 can include one or more chemical analysis devices to measure constituent species of the example flows shown in FIGS. 1 and 3.

In example implementations, flow control system 999 can include one or more temperature sensors (e.g., thermocouples, thermistors, thermometers) and temperature controllers to monitor and control one or more aspects of flow control system 999. In example implementations, flow control system 999 can include one or more power control units, to provide electrical power to components of the illustrated processes.

In example implementations, flow control system 999 can be operated manually. For example, an operator can set a flow rate for each pump and set valve open or close positions to regulate the flow of the process streams through the pipes in flow control system 999. Once the operator has set the flow rates and the valve open or close positions for all flow control systems 999 distributed across the illustrated processes, flow control system 999 can flow the streams under constant flow conditions, for example, constant volumetric rate or other flow conditions. To change the flow conditions, the operator can manually operate control system 999, for example, by changing the pump flow rate or the valve open or close position. An example two way valve 192 and three way valve 190 are shown in FIG. 1, while an example two way valve 392 and three way valve 390 are shown in FIG. 3.

In example implementations, flow control system 999 can be operated automatically. For example, the flow control system 999 can be connected to a computer or a computer-readable medium storing instructions (such as flow control instructions and other instructions) executable by one or more processors to perform operations (such as flow control operations). An operator can set the flow rates and the valve open or close positions for all flow control systems 999 distributed across the illustrated processes using the flow control system 999. In such implementations, the operator can manually change the flow conditions by providing inputs through the flow control system 999. Also, in such implementations, the flow control system 999 can automatically (that is, without manual intervention) control one or more of the flow control systems, for example, using feedback systems connected to flow control system 999. For example, a sensor (such as a pressure sensor, temperature sensor or other sensor) can be connected to a pipe through which a process stream flows. The sensor can monitor and provide a flow condition (such as a pressure, temperature, or other flow condition) of the process stream to flow control system 999. In response to the flow condition exceeding a threshold (such as a threshold pressure value, a threshold temperature value, or other threshold value), control system 999 can automatically perform operations. For example, if the pressure or temperature in the pipe exceeds the threshold pressure value or the threshold temperature value, respectively, flow control system 999 can provide a signal to the pump to decrease a flow rate, a signal to open a valve to relieve the pressure, a signal to shut down process stream flow, or other signals.

FIG. 2 is a schematic drawing of an example implementation of a membrane reactor that can be used in a process for membrane enhanced steam reforming with carbon dioxide utilization according to the present disclosure. FIG. 2, generally, is a simplified side-view representation of an electrically-heated catalytic membrane reactor 200 with the vessel wall depicted as a cross-section. Membrane reactor 200 can be implemented, for example, as an M-SMR, an M-WGSR, or both in the present disclosure.

The reactor 200 can be a reformer to convert an input flow 201 of a hydrocarbon or carbon monoxide (CO) (or combination thereof), an input flow 203 of nitrogen (N₂), and an input flow 205 of water (H₂O) to output flows 211 and 215 of carbon dioxide (CO₂) and water (H₂O) and an output flow 213 of nitrogen (N₂) and hydrogen (H₂). The catalytic membrane reactor 200, in some aspects, can include electrical resistive heaters and hydrogen-selective tubular membranes 204 in a vessel 202. The hydrogen-selective tubular membranes 204 can be hydrogen selective membranes with Palladium or a Palladium alloy.

The tubular membranes 204 can be characterized or labeled as cylindrical membranes, hollow membranes, and so on. The wall of the tubular membrane 204 is the membrane, i.e., the membrane material. A bore of each tubular membrane 204 is the interior cylindrical cavity (lumen) of the tubular membrane 204 and defined by the wall (membrane or membrane material) of the tubular membrane 204. The material of the hydrogen-selective tubular membranes 204 can be, for example, a palladium alloy. The membrane can be a thin film of palladium alloy supported on a tubular porous substrate composed of a metal or metal oxide.

In operation, hydrogen 209 can pass through the tubular membrane 204 wall from a catalyst bed 207 that receives the input fluids (that can be a mixture of water, carbon monoxide, and carbon dioxide). The bore of the tubular membrane 204 is the permeate side of the membrane 204. The permeate hydrogen can be collected as product from the bore. The vessel 202 volume space external to the tubular membranes 204 is the retentate side of the tubular membranes 204. The produced carbon dioxide can discharge from the vessel 202 from the retentate side.

As shown, the vessel 202 houses the tubular membranes 204 and (optionally) the resistive heaters. The vessel 202 can be, for example, stainless steel. The vessel 202 can be a cylindrical vessel. The vessel 202 can have a vertical orientation or a horizontal orientation.

In example aspects, the membrane reactor 200 as an M-SMR can include a metal based catalyst, such as nickel catalyst for natural gas steam reforming, with steam as a sweep gas (such as steam 125). In example aspects, the membrane reactor 200 as an M-WGSR can include a metal-oxide catalyst such as iron oxide catalyst or copper based catalyst.

FIG. 3 is a schematic drawing of another example implementation of a process 300 for membrane enhanced steam reforming with carbon dioxide utilization according to the present disclosure. Generally, as shown in this example, the process 300 is similar to the process 100 but instead of utilizing a dual M-SMR design with methanol production, process 300 utilizes a single M-SMR design without methanol production.

In process 300, residual carbon monoxide (CO) is converted in a membrane water gas shift reactor (M-WGSR). Oxygen used for the ATR is produced by splitting air (for example, atmospheric air) to oxygen and nitrogen in an air separation unit (ASU). Nitrogen produced in the ASU can be used as a sweep gas in an M-SMR as well as an M-WGSR. Generally, the reactors comprise the respective steam reforming or water gas shift reaction catalysts and each is integrated with a hydrogen selective membrane (as described with reference to FIG. 2 as well).

The process 300 includes process fluid flows that are circulated (naturally or forcibly or both) through the components of the process from left to right as shown in FIG. 3. Air 301 (for example, atmospheric air) is fed to an ASU 302 that separates the air 301 into oxygen 307 (O₂) and nitrogen 317 (N₂). The oxygen 307 is fed to an ATR 304 along with water 303 (H₂O) and a hydrocarbon 305 (such as, for example, natural gas 305). The nitrogen 317 is also provided to a first M-SMR 306, along with water 309 (H₂O) and a hydrocarbon 311 (such as natural gas). In this example, the nitrogen 317 is used as the sweep gas in the M-SMR 306.

The ATR 304, in this example, can be a combination of steam methane reforming (SMR) and partial oxidation (POₓ), where the reactor comprises a combustion section to generate the heat required for the endothermic reforming reaction, which is carried out in a catalyst bed in the ATR 304. The ATR product 319 (for example, a product of hydrogen, carbon monoxide, carbon dioxide, methane, and water) is further processed in the M-WGSR 310. The ATR product 319, in some aspects, can have mole fractions of: 0.51 hydrogen, 0.19 carbon monoxide, 0.05 carbon dioxide, 0.01 methane, and 0.25 water. Nitrogen 317 from the ASU 302 is also provided to the M-WGSR 310. The M-SMR 306 produces the combined flow 321 of H₂ and N₂, as well as carbon dioxide 323 (CO₂) and impurities 341.

As shown in FIG. 3, the combined hydrogen and nitrogen flows 353 and 321 combine into flow 351, which is fed to an ammonia synthesis unit 312, which produces ammonia 337 (NH₃). Ammonia 337 is fed to a urea synthesis unit 316 as well as output from the process 300 as shown.

The M-WGSR 310 produces carbon dioxide 335 (CO₂) as well as a combined flow 353 of hydrogen (H₂) and nitrogen (N₂). The M-WGSR 310 also produces impurities 339. The combined flow 353 combines with the combined flow 321 and feeds into the ammonia synthesis unit 312 as flow 351. The M-WGSR 310 feeds the carbon dioxide 335 to the urea synthesis unit 316. In some aspects, the M-WGSR 310 contains a tubular hydrogen separation palladium alloy membrane (shown in more detail in FIG. 2). The carbon dioxide 335 and water are obtained as retentate product in the M-WGSR 310, which are further used as feed for urea and methanol production as shown.

With the flows 323 and 335 of carbon dioxide, as well as the ammonia 337, the urea synthesis unit 316 produces urea 343 as an output of the process 300. As shown in FIG. 3, therefore, in this example implementation, the only primary outputs of process 300 are urea 343 and ammonia 337, with all carbon dioxide 335 and 323 being consumed in the process by stoichiometric balancing. For example, the stoichiometric balancing can include providing the ammonia 337 and carbon dioxide 335 and 323 (combined) to the urea synthesis unit 316 in a molar ratio in the range of between 1 and 6 (ammonia to carbon dioxide).

Secondary outputs such as impurities 339 and 341 are also produced. In this example, impurities can include carbon monoxide (CO) and methane (CH₄), which can be recycled back to the M-SMR 306 and the M-WGSR 310. In some aspects, the impurity composition on a molar basis for the recycled impurities can be 2% for methane and 1.7% for carbon monoxide.

In the example process 300, the nitrogen 317 is used as a sweep gas to the M-SMR 306 in a co-current/counter current mode of operation for further enhancement of hydrocarbon and carbon monoxide conversion. Hydrogen and nitrogen in combined flows 353 and 321 are obtained as a permeate stream (for example, in the ratio of 3:1) from the M-WGSR 310 and M-SMR 306, respectively, which are further processed in the ammonia synthesis unit 312 for ammonia production. The mole ratio of hydrogen and nitrogen in the combined flows 321 and 353 can be adjusted by changing the sweep gas flow or by changing other operating and design parameters (for example, membrane area, permeate pressure, feed pressure and temperature) to obtain a desired mole ratio for ammonia synthesis (for example, between 2.7 and 3.2).

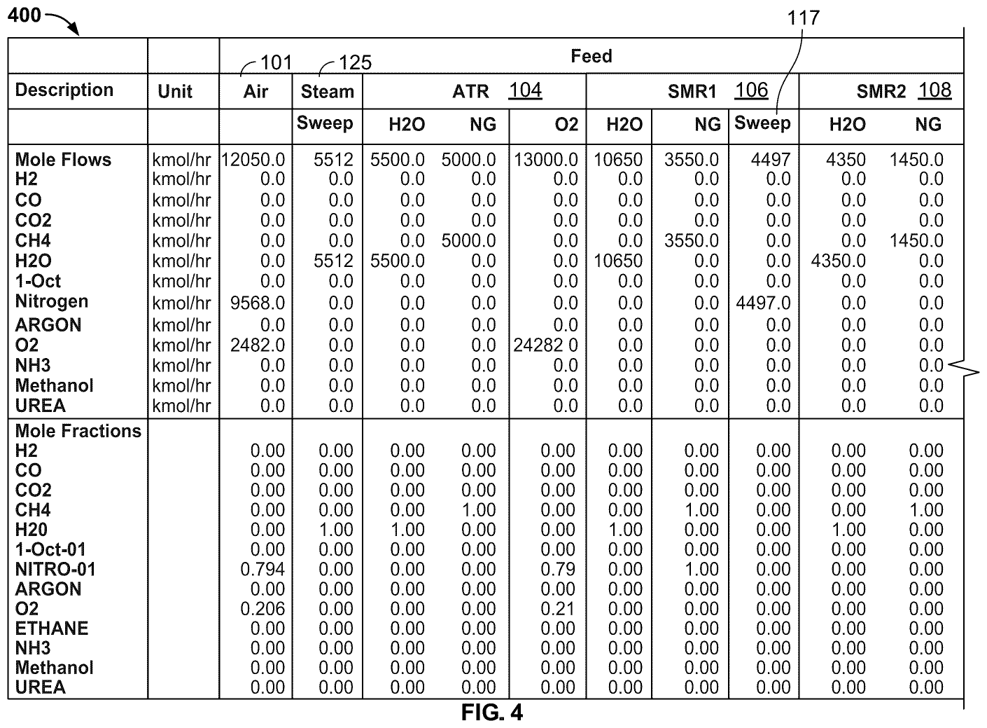

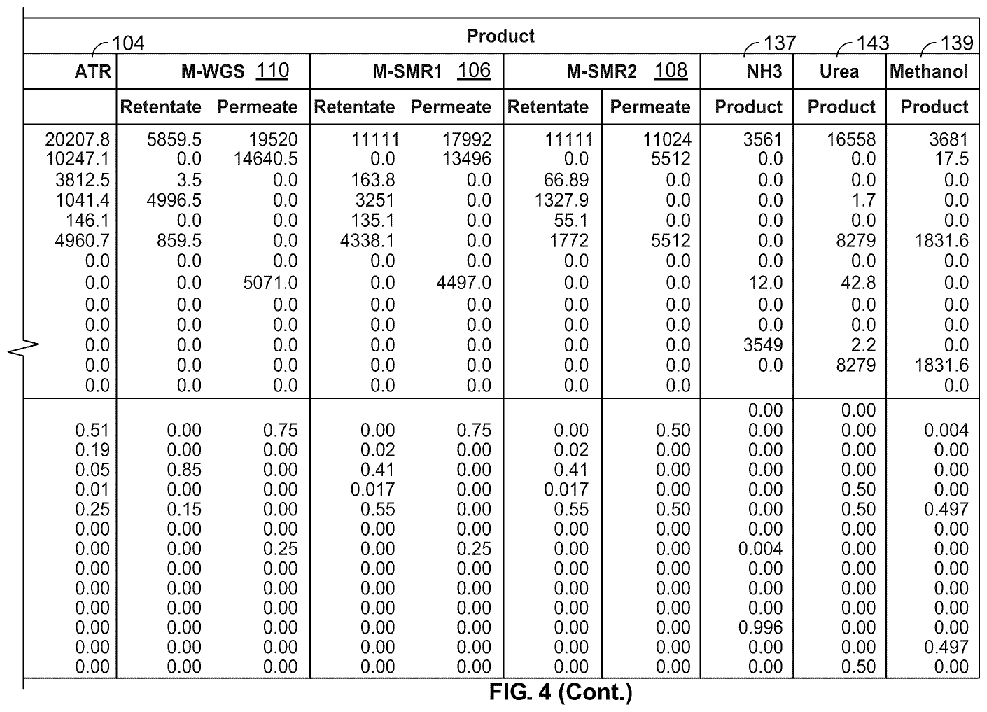

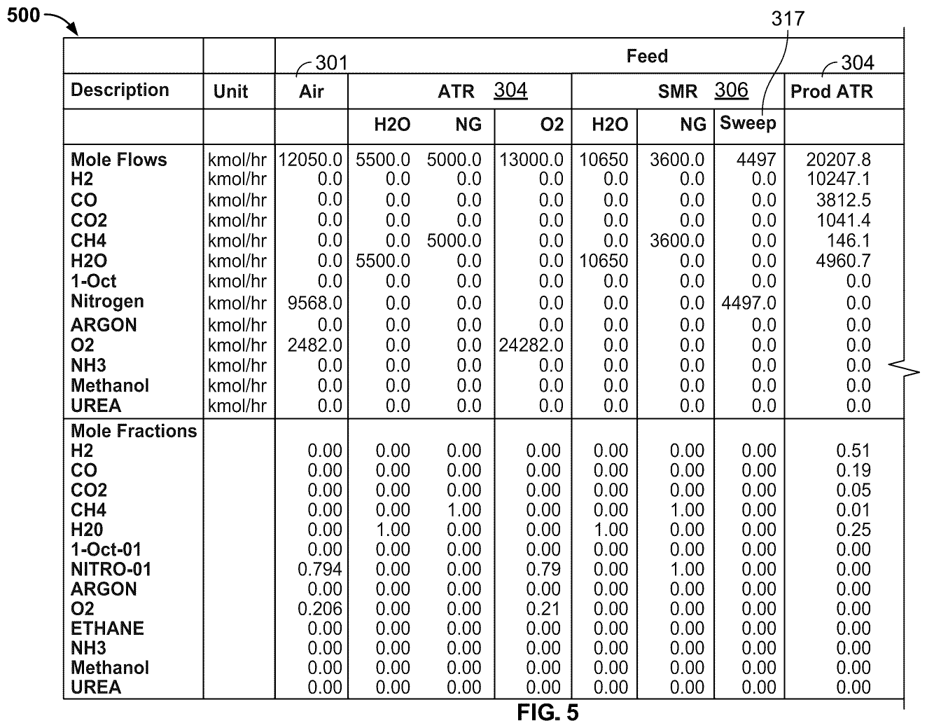

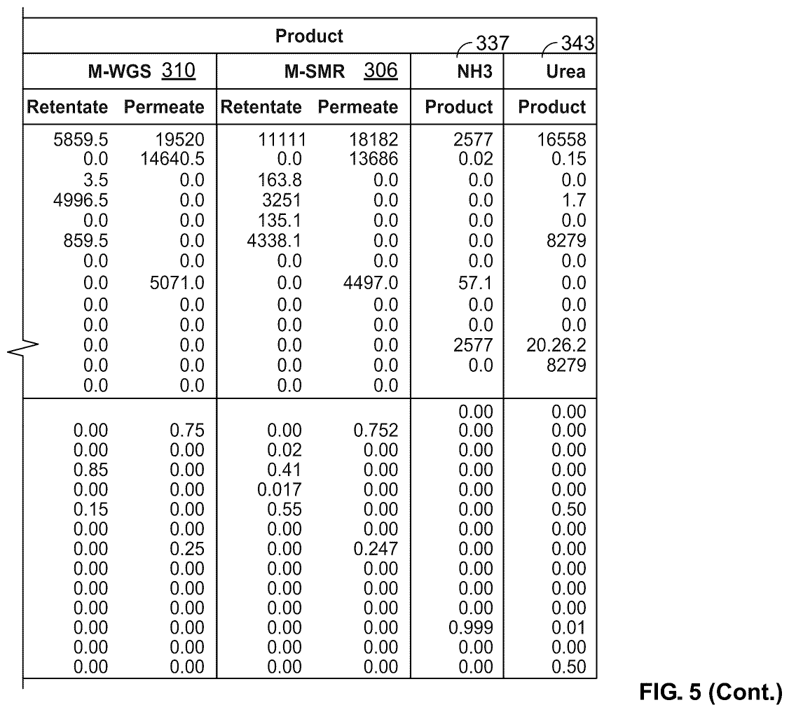

FIGS. 4 and 5 show tables 400 and 500, respectively, of example process mole flows and mole fractions of fluid streams in the respective processes of FIGS. 1 and 3, respectively, according to the present disclosure. For example, a process model for processes 100 and 300, respectively, was developed using pure methane as a feedstock (in other words, as the hydrocarbon) to generate the overall material balance. Tables 400 and 500 shows the mass balance for process 100, which incorporates ATR reforming, a membrane water gas shift reactor (M-WGS) and dual membrane reforming (M-SMR) (in Table 400 for process 100) or single M-SMR (in Table 500 for process 300) concepts.

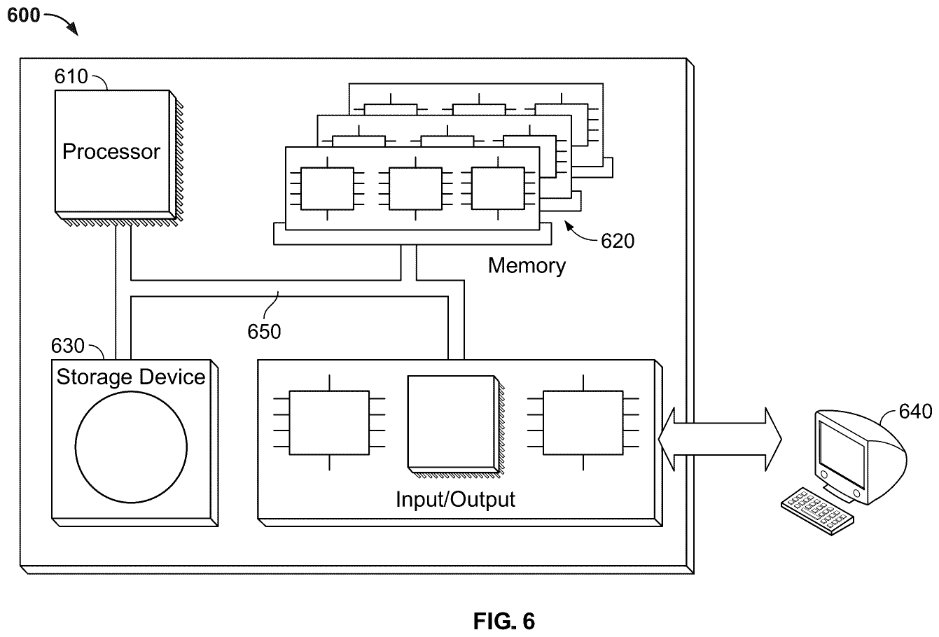

FIG. 6 is a schematic illustration of an example control system 600 for a process for membrane enhanced steam reforming with carbon dioxide utilization according to the present disclosure. For example, all or parts of the control system (or controller) 600 can be used for the operations described previously, for example as or as part of the flow control system 999. The controller 600 is intended to include various forms of digital computers, such as printed circuit boards (PCB), processors, digital circuitry, or otherwise. Additionally, the system can include portable storage media, such as, Universal Serial Bus (USB) flash drives. For example, the USB flash drives can store operating systems and other applications. The USB flash drives can include input/output components, such as a wireless transmitter or USB connector that can be inserted into a USB port of another computing device.

The controller 600 includes a processor 610, a memory 620, a storage device 630, and an input/output device 640. Each of the components 610, 620, 630, and 640 are interconnected using a system bus 650. The processor 610 is capable of processing instructions for execution within the controller 600. The processor can be designed using any of a number of architectures. For example, the processor 610 can be a CISC (Complex Instruction Set Computers) processor, a RISC (Reduced Instruction Set Computer) processor, or a MISC (Minimal Instruction Set Computer) processor.

In one implementation, the processor 610 is a single-threaded processor. In another implementation, the processor 610 is a multi-threaded processor. The processor 610 is capable of processing instructions stored in the memory 620 or on the storage device 630 to display graphical information for a user interface on the input/output device 640.

The memory 620 stores information within the control system 600. In one implementation, the memory 620 is a computer-readable medium. In one implementation, the memory 620 is a volatile memory unit. In another implementation, the memory 620 is a non-volatile memory unit.

The storage device 630 is capable of providing mass storage for the controller 600. In one implementation, the storage device 630 is a computer-readable medium. In various different implementations, the storage device 630 can be a floppy disk device, a hard disk device, an optical disk device, a tape device, flash memory, a solid state device (SSD), or a combination thereof.

The input/output device 640 provides input/output operations for the controller 600. In one implementation, the input/output device 640 includes a keyboard and/or pointing device. In another implementation, the input/output device 640 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, for example, in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a unit, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, solid state drives (SSDs), and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) or LED (light-emitting diode) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a control system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

While this specification contains many specific implementation details, these should not be construed as limitations on the scope of any inventions or of what can be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features can be described above as acting in certain combinations and even initially claimed as such, one or more features from a claimed combination can in some cases be excised from the combination, and the claimed combination can be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing can be advantageous. Moreover, the separation of various system components in the implementations described above should not be understood as requiring such separation in all implementations, and it should be understood that the described program components and systems can generally be integrated together in a single software product or packaged into multiple software products.

A number of implementations have been described. Nevertheless, it will be understood that various modifications can be made without departing from the spirit and scope of the disclosure. For example, example operations, methods, or processes described herein can include more steps or fewer steps than those described. Further, the steps in such example operations, methods, or processes can be performed in different successions than that described or illustrated in the figures. Accordingly, other implementations are within the scope of the following claims.

What is claimed is:

1. A process, comprising:
feeding a flow atmospheric air to an air separation unit to produce a flow of nitrogen and a flow of oxygen;
combining the flow of oxygen with a hydrocarbon flow and a flow of water in an auto-thermal reformer to produce a retentate stream to a membrane water gas shift reactor (M-WGSR);
generating, from the retentate stream to the M-WGSR, a permeate stream from the M-WGSR that comprises a first flow of carbon dioxide and a first combined flow of hydrogen and nitrogen;
feeding a retentate stream to a membrane steam methane reformer (M-SMR) to produce a permeate stream from the M-SMR that comprises a second flow of carbon dioxide and a second combined flow of hydrogen and nitrogen;
feeding the first and second combined flows of hydrogen and nitrogen to an ammonia synthesis unit to produce a flow of ammonia; and
feeding the first and second flows of carbon dioxide and the flow of ammonia to a urea synthesis unit to produce a flow of urea by fully utilizing the first and second flows of carbon dioxide.

2. The process of claim 1, wherein the retentate stream to the M-SMR comprises another flow of water and another hydrocarbon flow.

3. The process of claim 2, wherein the M-SMR is a first M-SMR, the process further comprising:
feeding a retentate stream to a second M-SMR to produce a permeate stream from the second M-SMR that comprises a flow of hydrogen and a third flow of carbon dioxide.

4. The process of claim 3, further comprising:
combining the flow of hydrogen with the flow of nitrogen from the air separation unit into a third combined flow of hydrogen and nitrogen; and
feeding the third combined flow of hydrogen and nitrogen to the ammonia synthesis unit to produce the flow of ammonia.

5. The process of claim 3, further comprising feeding a portion of the third flow of carbon dioxide from the second M-SMR to the urea synthesis unit to produce the flow of urea by fully utilizing the first, second, and portion of the third flows of carbon dioxide.

6. The process of claim 5, further comprising feeding another portion of the third flow of carbon dioxide from the second M-SMR and the flow of hydrogen from the second M-SMR to a methanol synthesis unit to produce a flow of methanol.

7. The process of claim 3, further comprising feeding a flow of steam to the second M-SMR as a sweep gas to produce the permeate stream from the second M-SMR.

8. The process of claim 1, further comprising:
feeding a portion of the flow of nitrogen to the M-SMR as a sweep gas to produce the permeate stream from the M-SMR; and
feeding another portion of the flow of nitrogen to the M-WGSR as a sweep gas to produce the permeate stream from the M-WGSR.

9. The process of claim 1, further comprising outputting a portion of the flow of ammonia.

10. The process of claim 1, further comprising, in each of the M-SMR and the M-WGSR, utilizing a hydrogen selective membrane to produce the respective permeate streams from the M-SMR and M-WGSR.

11. A system, comprising:
an air separation unit;
an auto-thermal reformer in fluid communication with the air-separation unit;
a membrane water gas shift reactor (M-WGSR) fluidly coupled to the air separation unit and the auto-thermal reformer;
a membrane steam methane reformer (M-SMR) fluidly coupled to the air separation unit;
an ammonia synthesis unit fluidly coupled to the M-WGSR and M-SMR;
a urea synthesis unit fluidly coupled to the M-WGSR, the M-SMR, and the ammonia synthesis unit; and
a flow control system configured to perform operations, comprising:
feeding a flow atmospheric air to the air separation unit to produce a flow of nitrogen and a flow of oxygen;
combining the flow of oxygen with a hydrocarbon flow and a flow of water in the auto-thermal reformer to produce a retentate stream to the M-WGSR;
generating, from the retentate stream to the M-WGSR, a permeate stream from the M-WGSR that comprises a first flow of carbon dioxide and a first combined flow of hydrogen and nitrogen;
feeding a retentate stream to the M-SMR to produce a permeate stream from the M-SMR that comprises a second flow of carbon dioxide and a second combined flow of hydrogen and nitrogen;
feeding the first and second combined flows of hydrogen and nitrogen to the ammonia synthesis unit to produce a flow of ammonia; and
feeding the first and second flows of carbon dioxide and the flow of ammonia to the urea synthesis unit to produce a flow of urea by fully utilizing the first and second flows of carbon dioxide.

12. The system of claim 11, wherein the retentate stream to the M-SMR comprises another flow of water and another hydrocarbon flow.

13. The system of claim 12, wherein the M-SMR is a first M-SMR, the system comprises a second M-SMR, and the operations further comprise:
feeding a retentate stream to the second M-SMR to produce a permeate stream from the second M-SMR that comprises a flow of hydrogen and a third flow of carbon dioxide.

14. The system of claim 13, wherein the operations further comprise:
combining the flow of hydrogen with the flow of nitrogen from the air separation unit into a third combined flow of hydrogen and nitrogen; and
feeding the third combined flow of hydrogen and nitrogen to the ammonia synthesis unit to produce the flow of ammonia.

15. The system of claim 13, wherein the operations further comprise feeding a portion of the third flow of carbon dioxide from the second M-SMR to the urea synthesis unit to produce the flow of urea by fully utilizing the first, second, and portion of the third flows of carbon dioxide.

16. The system of claim 15, wherein the operations further comprise feeding another portion of the third flow of carbon dioxide from the second M-SMR and the flow of hydrogen from the second M-SMR to a methanol synthesis unit to produce a flow of methanol.

17. The system of claim 13, wherein the operations further comprise feeding a flow of steam to the second M-SMR as a sweep gas to produce the permeate stream from the second M-SMR.

18. The system of claim 11, wherein the operations further comprise:
feeding a portion of the flow of nitrogen to the M-SMR as a sweep gas to produce the permeate stream from the M-SMR; and
feeding another portion of the flow of nitrogen to the M-WGSR as a sweep gas to produce the permeate stream from the M-WGSR.

19. The system of claim 11, wherein the operations further comprise outputting a portion of the flow of ammonia.

20. The system of claim 11, wherein each of the M-SMR and the M-WGSR comprises a hydrogen selective membrane configured to produce the respective permeate streams from the M-SMR and M-WGSR.
